(19) United States
(12) Patent Application Publication
(10) Pub. No.: US 2021/0128055 A1
(43) Pub. Date: May 6, 2021

(54) METHOD AND DEVICE FOR DETERMINING SKIN MOISTURE CONTENT

(71) Applicant: Henkel AG & Co. KGaA, Duesseldorf (DE)

(72) Inventors: ANDREAS BOCK, Nuess (DE); THOMAS WELSS, Duesseldorf (DE); CLAUDIA HUNDEIKER, Meerbusch (DE); MARIANNE WALDMANN-LAUE, Monheim (DE)

(73) Assignee: Henkel AG & Co. KGaA, Duesseldorf (DE)

(21) Appl. No.: 16/493,516

(22) PCT Filed: Feb. 16, 2018
(86) PCT No.: PCT/EP2018/053864

§ 371 Date: Sep. 12, 2019

(30) Foreign Application Priority Data
Mar. 17, 2017 (DE) 10 2017 204 491.8

(51) Int. Cl. A61B 5/00; A61B 5/0531

(52) U.S. Cl. CPC A61B 5/443; A61B 5/0022; A61B 5/6801; A61B 5/0531

(57) ABSTRACT

In various embodiments, a method for determining a skin moisture content is provided. The method includes: contacting at least one skin region with at least one sensor contact region of a skin moisture measuring device; detecting a signal dependent on skin moisture in the skin region with the skin moisture measuring device; and associating a skin moisture content with the detected signal by comparing the detected signal with reference signals for known skin moisture contents, wherein the reference signals are located in a database stored in a processor cloud architecture.

### CROSS-REFERENCE TO RELATED APPLICATION

This application is a U.S. National-Stage entry under 35 U.S.C. § 371 based on International Application No. PCT/EP2018/053864, filed Feb. 16, 2018, which was published under PCT Article 21(2) and which claims priority to German Application No. 10 2017 204 491.8, filed Mar. 17, 2017, which are all hereby incorporated in their entirety by reference.

### TECHNICAL FIELD

Various embodiments generally relate to a method and device for determining skin moisture content and to a method for determining a cosmetic skin treatment recommendation.

### BACKGROUND

A balanced supply of skin with moisture can be crucial for a youthful radiant appearance of the skin. A content of water bound in the skin (also referred to as skin moisture content) can steadily decrease with age. The skin can then appear dry and limp and lose its radiance.

The skin moisture content can be improved by a variety of different technologies by means of skin care products. In particular, technologies can be physiologically relevant in which active substances penetrate into the skin and optimize binding of water in the epidermis and the dermis.

A possibility of a timely detection of deficiencies in the skin moisture can enable the user to react immediately to a deficiency situation.

Without expert dermatological or cosmetic advice, it can be difficult for a user to determine his individual skin condition (for example, skin moisture content) and cosmetics suitable for their skin condition (for example, skin moisture content).

If the user still consumes cosmetic products, it can be virtually impossible to reconstruct a treatment success, because the user, for example, at home, lacks opportunities to assess a treatment outcome in a standardized and objective manner.

As a result, it can be difficult for the consumer to assess the individual effectiveness of a cosmetic, which can result in a motivation to perform a corresponding cosmetic treatment, for example, in the longer term, being impaired. This can be the case even if a cosmetic product were capable of achieving an objectively detectable desired effect.

In many areas of daily life, there has been a trend towards personalized programs that can respond to individual requirements and needs, for example, in a nutrition or health area, but also in an area of personalized cosmetics. This can enable a user to find targeted cosmetic products and/or to obtain care instructions that are tailored to the individual needs of his skin, and thus enable a particularly high effectiveness.

A device and method for determining skin moisture content can be provided in various embodiments. The determination of the skin moisture content in the skin and a continuous checking of changes can offer the user an added benefit of an individualizable treatment in various embodiments.

### BRIEF SUMMARY

A method for determining a skin moisture content is provided in an embodiment herein. The method includes: contacting at least one skin region with at least one sensor contact region of a skin moisture measuring device; detecting a signal dependent on skin moisture in the skin region with the skin moisture measuring device; and associating a skin moisture content with the detected signal by comparing the detected signal with reference signals for known skin moisture contents, wherein the reference signals are located in a database stored in a processor cloud architecture.

In another embodiment, a device for determining a skin moisture content is provided. The device include: a skin moisture measuring device having at least one sensor contact region and configured to provide a signal dependent on a skin moisture in the skin region by contacting at least one skin region of a user with the at least one sensor contact region; an electronic circuit device configured for detecting the signal provided by the skin moisture measuring device and associating a skin moisture content with the detected signal by comparing the detected signal with reference signals for known skin moisture contents, wherein association of the skin moisture content with the detected signal is performed with a database stored in a processor cloud architecture.

In another embodiment, a device for determining a skin moisture content is provided. The device includes a breathable carrier; a skin moisture measuring device arranged on the breathable carrier, wherein the skin moisture measuring device includes at least one sensor contact region configured to provide a signal dependent on a skin moisture in the skin region by contacting of at least one skin region of a user with the at least one sensor contact region; and an electronic circuit device arranged on the breathable carrier for detecting the signal provided by the skin moisture measuring device and for associating a skin moisture content with the detected signal by comparing the detected signal with reference signals for known skin moisture contents.

In various embodiments, a sensor which detects a content of water in the skin (more precisely, a sensor measurement can be detected and the skin moisture content can be determined therefrom) can be used to determine the skin moisture content of the skin (for example, in particular the epidermis and dermis). A special device can be used for this purpose, which device can be connected to or integrated into a smartphone, a tablet or another data processing device (for example, a computer).

In various embodiments, the sensor for determining the skin moisture (also referred to as a skin moisture measuring device) for the measurement can use, for example, an impedance, a dielectric constant, an electrical conductance or a thermal conductivity of the skin. In this case, a measuring principle of the skin moisture measuring device can be known in principle, for example, a corneometer can be used to determine the dielectric constant of the skin (rising with greater water content), or, for example, a so-called TTT device (TTT stands for “transient thermal transfer”) can be used to determine the thermal conductivity of the skin (also increasing with greater water content). In various embodiments, the skin moisture measuring device can have a sensor contact region that requires contact with the skin to determine its moisture content.

In various embodiments, the skin moisture measuring device (sensor) can be attached to a carrier. The carrier can be designed in various embodiments as a patch, that is, as a flat flexible (for example, bendable) carrier, which is provided on one side with an adhesive with which the patch can be fastened to the skin of the user. Thus, a continuous measurement and/or a repeated measurement over a longer period of time (for example, several times within minutes, hours, days or weeks) may be possible.

The skin moisture content can be determined in various embodiments from the measured value (also referred to as a measurement signal or signal), for example, by means of an electronic circuit device.

In various embodiments, the detected skin moisture content can be determined by means of an app or other software and provided in the form of a value (for example, using arbitrary units), as a graphical representation and/or acoustic message.

In various embodiments, even when the carrier is not formed as a patch, the skin moisture content can be recorded over a long period of time (for example, minutes, hours, days or similar).

In various embodiments, a consequence of a dysbalance can be described on the basis of the skin moisture content.

In various embodiments, an individualized cosmetic treatment to optimize the condition can be recommended based on the skin moisture content.

In various embodiments, the device can be formed convenient and small (for example, wearable). The device can be applied to a (live) user, for example, the user at home, at a point of sale for cosmetics, at a dermatologist and/or at a beauty salon. In various embodiments, the device can have a smartphone, a tablet, an iPad or similar electronic circuit device (for example, a data processing device and/or a data transfer device), and an integrated device or an additional device that is attachable, for example, to the electronic circuit device, or connectable thereto (for example, connectable by means of a data connection). The integrated device or additional device (possibly even the entire device) can have a size which enables it to be easily accommodated in a hand or trouser pocket, for example, having an area less than about 36 cm² and having a thickness less than about 2 cm.

In various embodiments, the signal determined by means of the skin moisture measuring device can be compared with reference signals for known skin moisture contents and from which a skin moisture content of the user can be determined. In various embodiments, the associated skin moisture contents can be stored in a database, for example, in an external data processing device, for example, a processor cloud architecture, wherein the comparison can be made, for example, such that database entries for the reference signal are directly compared with the signal, or, for example, such that the signal is converted to a skin moisture content of the user by means of a relationship determined on the basis of reference signals and reference skin moisture contents. The reference data (reference signals, associated reference skin moisture contents) can have been previously determined in various embodiments, for example, in laboratory experiments, wherein the reference skin moisture contents can have been determined, for example, in a conventional (for example, expensive) manner. Furthermore, when storing the database in the processor cloud architecture, in various embodiments, the database can be updated based on new associations of the skin moisture content with the detected signal from a plurality of further users. The updating can take place, for example, at regular intervals, for example, predetermined intervals, and/or, for example, whenever one of the further users provides one of the new associations.

In various embodiments, comparing the signal with the reference signal and/or determining the skin moisture content therefrom can be done by means of the electronic circuit device (for example, directly), for example, by means of an app or other software program which is operated by the electronic circuit device (for example, a smartphone, a tablet, an iPad or similar).

In various embodiments, comparing the signal with the reference signal and/or determining the skin moisture content therefrom can be done indirectly by means of the electronic circuit device, for example, by providing the electronic circuit device with a (for example, contactless) data communication connection and by providing by means of the data communication connection the signal and/or result of the comparison of the signal with the reference signal of an external data processing device (for example, the processor cloud architecture) and by receiving from the external data processing device the skin moisture content determined by means of the signal and/or the result of the comparison.

In various embodiments, the skin moisture measuring device can have at least one sensor contact region, which can be arranged to be brought into contact with the skin (for example, a skin region of the user) for measuring skin moisture. For example, electrodes of a capacitor, electrodes for applying an alternating voltage or heat probes for introducing a quantity of heat into the skin and for measuring an increase in temperature of the skin can be in the sensor contact region.

In various embodiments, in particular when provided, the device for determining skin moisture content for a prolonged period of time (for example, a period of time longer than required for a single measurement, for example, longer than about one second, thus, for example, several seconds, one or more minutes, or even hours or days) can be left on the skin of the user, for example, for repeated measurements or a continuous measurement of the skin moisture content, a region of the sensor contact region can be minimized, for example, designed as small as is possible with the type of skin moisture measuring device used. In various embodiments, the sensor contact region can have a maximum area of about 1 mm², for example, a maximum of about 0.5 mm² or less.

In various embodiments, the carrier and/or other environment of the sensor contact region can be configured such that accumulation of moisture released from the skin between the sensor contact region and the skin is avoided or at least mitigated. In various embodiments, the carrier can be configured breathable (for example, as a breathable patch), for example, by means of openings extending from the skin and/or from above the sensor through the carrier to outside the device for determining skin moisture, wherein the openings are can be permeable to steam (for example, water vapor) and/or for liquids (for example, water, sweat). In various embodiments, a moisture-absorbing material can be arranged adjacent to the sensor contact region, for example, such that the skin region contacted by the sensor contact region and an adjacent skin region, which is contacted by the moisture-absorbing material, directly adjoin one another. The moisture-absorbing material can be arranged, for example, surrounding the sensor contact region.

In various embodiments, the design that mitigates or avoids the accumulation of moisture between the sensor contact region and the skin can enable repeated determination of the skin moisture content in the skin over a longer time period and/or continuously without the measurement results being falsified after a short time (for example, after several seconds or a few minutes) by moisture emitted from the skin and then resting on the skin as a liquid film.

In various embodiments, to improve a signal-to-noise ratio, an overall sensor contact region can be increased by providing a plurality of sensor contact regions spaced apart from one another. A signal can be provided by means of each of the sensor contact regions, wherein in various embodiments, an average value can be formed from the plurality of signals. In various embodiments, the moisture-absorbing material can be arranged in a spacing region between the sensor contact regions.

In various embodiments, the determined skin moisture content can be provided to the user in any manner, for example, for the user, for example, as a numerical value (in arbitrary or physical units, for example, as weight percentage, for example, in percent), a graphical representation (for example, a representation of the determined value in relation to a total value range from very low in moisture to normal to very moist), an acoustic message, or similar.

In particular, in a case in which the skin moisture content is determined for a plurality of skin regions, providing the skin moisture content to the user can have a graphical representation, for example, a display, for example, by means of a display device (for example, a display of a smartphone, tablet, iPad, or smart mirrors). The determined skin moisture content of the plurality of regions can be displayed in the graphical representation, using a coding based on the skin moisture content in a representation of the user (for example, a schematic representation or on a photo of the user). For example, in a schematic representation of a user's face, regions of different skin moisture content can be displayed with different colors and/or patterns, for example, regions having normal skin moisture content green, having low skin moisture content red and having high skin moisture content blue or similar. In another example, a user's photograph, for example, a digital photograph showing the user's face, can be overlaid with different patterns, for example, a dot pattern for regions having high skin moisture content and a line pattern for regions having low skin moisture, wherein regions of normal skin can be left unpatterned, or similar. Alternatively, the real-time representation of a body or body region (for example, the face) on/in a smart mirror can be used to represent the determined skin moisture contents.

When graphically illustrating the skin moisture content of the at least one skin region, for example, the plurality of the skin regions, the skin moisture content can be represented in various embodiments only for the region(s) for which the skin moisture content was determined. In various embodiments, a region for which a determined skin moisture content is displayed can be extrapolated beyond the skin region for which the skin moisture content was determined, for example, including typical skin moisture distribution patterns.

In various embodiments, information about where the region(s) is/are can be provided to the electronic circuit device, for example, by means of the user, for example, by means of an input device (for example, tapping a touch-sensitive display on which a schematic or photographic representation of the user is displayed, by means of text input via a keyboard or any other suitable input option).

In various embodiments, the display device can be part of the device, for example, a display of a smartphone, tablet or iPad. In various embodiments, the display device can be coupled to the device, for example, by means of a (for example, wireless) data connection.

Additionally or alternatively, in various embodiments, a quality value can be associated with the determined skin moisture content, with a high quality value for normal/healthy skin and a low quality value for a moisture-deficient or excessively moisturized skin, wherein the problem is more likely to be dehydration than over hydration.

In various embodiments, a user is to be enabled to find targeted cosmetic products and/or to obtain care suggestions that can be tailored to the individual needs of the user's skin.

In various embodiments, at least one cosmetic product can be recommended to the user based on the determined skin moisture content. The cosmetic product can be suitable for maintaining or improving a skin moisture content of the user (for example, normalizing or stabilizing a skin moisture content in a normal condition). In various embodiments, the cosmetic product can have a cream, a lotion, an ointment, an oil, an emulsion, a gel, a soap, a mask, an ampoule having an adjuvant care product, a toner, a serum, a spray, or similar.

The normalization or stabilization of a skin moisture content by a cosmetic product can be done with the aid of moisturizing and/or moisture-binding ingredients. Moisturizing and/or moisture-binding ingredients comprise in particular aloe vera, vegetable oils such as hemp oil or evening primrose oil, unsaturated fatty acids, urea, glycerol and/or dexpanthenol.

In various embodiments, the product recommendations and/or care instructions can be provided, for example, by means of software, for example, an app. For example, the user can be advised to apply a cosmetic care product which moisturizes to the at least one skin region having low skin moisture content, and/or to drink much (for example, water).

In various embodiments, the software/app that determines the skin moisture content can be the same as that which determines the product recommendation and/or the care instructions. In various embodiments, different software programs/apps can be used for a part of the various operations or all of the various operations (determining the skin moisture content, determining a product recommendation, determining a care instruction).

In various embodiments, a treatment success in a cosmetic treatment, which can have as a goal a positive effect on the determined skin moisture content, can be monitored. In various embodiments, the software/app can enable control and/or tracking of the results by means of a representation (for example, a graphical representation) of the measurement results over time.

In various embodiments, when determining the product and care recommendations, further information can also be used regarding general health, skin condition, dietary habits, and further user behaviors (for example, daily exposure to outdoors, sun, water, smoking habits, sports habits, etc.), for example, by means of the software/app. This information can be requested by the user in various embodiments by means of the software/app.

In various embodiments, literature data can be the basis of assessing suitability of a care product and/or a care instruction in caring for a skin having a given skin moisture content.

In various embodiments, each skin moisture content of a plurality of skin moisture contents can be associated with a quality score. A care product and/or a care instruction can be rated suitable for a skin moisture content, when it is expected, for example, based on literature data, experimental results, or empirical data (which can, for example, be continually updated, for example, during use of the software/app by the user, with new empirical values of other users, see below), that the skin moisture content of the user is maintained or changed to a skin moisture content with a higher (that is, better) quality value in the case of (for example, regular) application of the care product and/or the care instruction.

In various embodiments, an assessment of a suitability of a care product to improve a skin moisture content can be confirmed or modified by means of receiving empirical values of further users having the same or a similar skin moisture content, for example, empirical values with regard to treatment success. Preferably, the further users also have, in addition to the same or a similar skin moisture content, the same or similar profile with respect to age, sex, general health, general skin condition, apart from skin moisture content and/or behaviors such as smoking, diet and/or exercise. It is thus possible for the user to always receive an optimal recommendation.

In various embodiments, it can be possible to check and track the effectiveness of a cosmetic treatment in an objective and standardized manner. The cosmetic treatment can, in various embodiments, have the goal of normalizing a skin moisture content.

In various embodiments, an efficacy of a (for example, cosmetic) treatment can be better understood and a selection of an individually suitable product can thereby be simplified.

In various embodiments, a user's motivation can be increased to perform a cosmetic treatment in the longer term, for example, by means of a comparison with other users, for example, by means of information about treatment successes provided by the other users.

In various embodiments, a method for determining a skin moisture content is provided. The method can have a contacting of the at least one skin region for at least one skin region of the user by means of at least one sensor contact region of a skin moisture measuring device, a detecting of a signal dependent on skin moisture in the skin area provided by means of the skin moisture measurement device and an associating of a skin moisture content with the detected signal by means of comparing the detected signal with reference signals for known skin moisture content, wherein the associating of the skin moisture content with the detected signal can take place by means of a database stored in a processor cloud architecture.

In various embodiments, the method can further have updating the database based on new association of the skin moisture content to the detected signal from a plurality of further users.

In various embodiments, the skin moisture measuring device can have at least one of a group of skin moisture measuring devices, wherein the group can have a capacitive skin moisture measuring device, an impedance skin moisture measuring device, a skin moisture measuring device utilizing the electrical conductance of the skin, and a skin moisture measuring device utilizing transient heat transfer of the skin.

In various embodiments, after contacting the at least one skin region by means of the at least one sensor contact region of the skin moisture measuring device, the detection of the signal and the association of the skin moisture content with the detected signal can be carried out several times at a time interval or continuously.

In various embodiments, the at least one sensor contact region can have a plurality of sensor contact regions spaced apart from one another, and the method can further have a forming of a signal average value from the plurality of signals of the plurality of sensor contact regions.

In various embodiments, the at least one skin region can have a plurality of skin regions of the user.

In various embodiments, the skin moisture measuring device can be part of a wearable device.

In various embodiments, the wearable device can have a smartphone, a tablet, or an iPad, or can be attachable to a smartphone, a tablet, or an iPad.

In various embodiments, the detection of the signal and/or the association of the skin moisture content with the detected signal can take place by means of an app.

In various embodiments, a method for determining a cosmetic treatment recommendation is provided. The method can have a determining of a skin moisture content according to various embodiments and a determining of the cosmetic product and/or a care instruction based on the determined skin moisture content and a further database stored in the processor cloud architecture, having a plurality of skin moisture contents and a plurality of associated cosmetic products and/or care instructions, wherein each skin moisture content of the plurality of skin moisture contents is associated with at least one suitable cosmetic product and/or at least one care instruction.

In various embodiments, the cosmetic product and/or the care instruction can be suitable for the skin moisture content when normalization of the skin moisture content is to be expected based on empirical values of a plurality of further users with the cosmetic product stored in the further database.

In various embodiments, determining the skin moisture content and/or determining the cosmetic skin treatment recommendation can have a transmitting of the signal and/or the skin moisture content to an external data processing device and a receiving of the skin moisture content and/or the cosmetic skin treatment recommendation.

In various embodiments, a device for determining a skin moisture content is provided. The device can have a skin moisture measuring device, which can have at least one sensor contact region and be set up to provide a signal dependent on a skin moisture in the skin region when contacting at least one skin region of a user by means of the at least one sensor contact region, an electronic circuit device for detecting the signal provided by the skin moisture measuring device and associating a skin moisture content with the detected signal by means of comparing the detected signal with reference signals for known skin moisture contents, wherein the associating of the skin moisture content with the detected signal takes place by means of a database stored in a processor cloud architecture.

In various embodiments, the skin moisture measuring device can have at least one of a group of skin moisture measuring devices, wherein the group can have a capacitive skin moisture measuring device, an impedance skin moisture measuring device, a skin moisture measuring device utilizing the electrical conductance of the skin, and a skin moisture measuring device utilizing transient heat transfer of the skin.

In various embodiments, the at least one sensor contact region can have an area of less than about 1 mm².

In various embodiments, the device can further have a breathable carrier, wherein the skin moisture measuring device and the electronic circuit device are arranged on the breathable carrier.

In various embodiments, the breathable carrier can be configured as an adhesive patch with the skin moisture measuring device and the electronic circuit device.

In various embodiments, the device can further have a wireless data interchange device arranged on the breathable support for exchanging data between the electronic circuit device and the processor cloud architecture.

In various embodiments, a device for determining a skin moisture content is provided. The device can have a breathable support, a skin moisture measuring device that can be arranged on the breathable carrier and at least one sensor contact region and set up to provide, upon contacting at least one skin region of a user by means of the at least one sensor contact region, a signal dependent on skin moisture in the skin region and an electronic circuit device arranged on the breathable support for detecting the signal provided by the skin moisture measuring device and for associating a skin moisture content with the detected signal by means of comparing the detected signal with reference signals for known skin moisture contents.

In various embodiments, the breathable carrier can be configured as an adhesive patch with the skin moisture measuring device and the electronic circuit device.

### BRIEF DESCRIPTION OF THE DRAWINGS

In the drawings, like reference characters commonly refer to the same parts throughout the several views, wherein for the sake of clarity, partial omission is made to provide reference numerals to all corresponding parts throughout the figures. Parts of the same or similar type may be provided with a trailing digit or a trailing small letter to distinguish them in addition to a common reference numeral. The drawings are not necessarily intended to depict scale, but rather the emphasis is on illustrating the principles of the present disclosure. In the following description, various embodiments as contemplated herein are described with reference to the following drawings, in which:

FIG. 1 shows a device for determining skin moisture content according to various embodiments and illustrates a use of the device;

FIG. 2A and FIG. 2B each show a device for determining a skin moisture content according to various embodiments;

FIG. 3A, FIG. 3B and FIG. 3C each show a device for determining a skin moisture content according to various embodiments and illustrate a use of the device;

FIG. 4 illustrates skin regions for applying a method and device for determining skin moisture content according to various embodiments;

FIG. 5 shows a flow chart illustrating a method for determining a skin treatment recommendation according to various embodiments;

FIG. 6 shows a flow chart of a method for determining a skin moisture content according to various embodiments; and

FIG. 7 shows a flowchart of a method for determining a cosmetic skin treatment recommendation according to various embodiments.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The term “exemplary” is used herein to mean “serving as an example, exemplar or illustration.” All embodiments or designs described herein as “exemplary” are not necessarily to be interpreted as preferred or advantageous over other embodiments or designs.

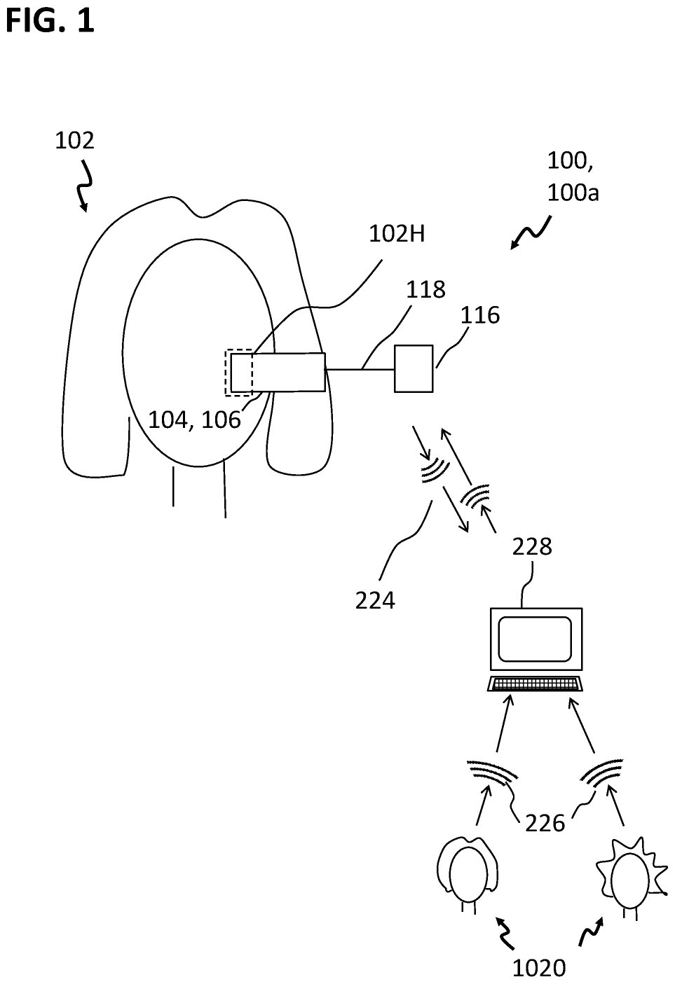

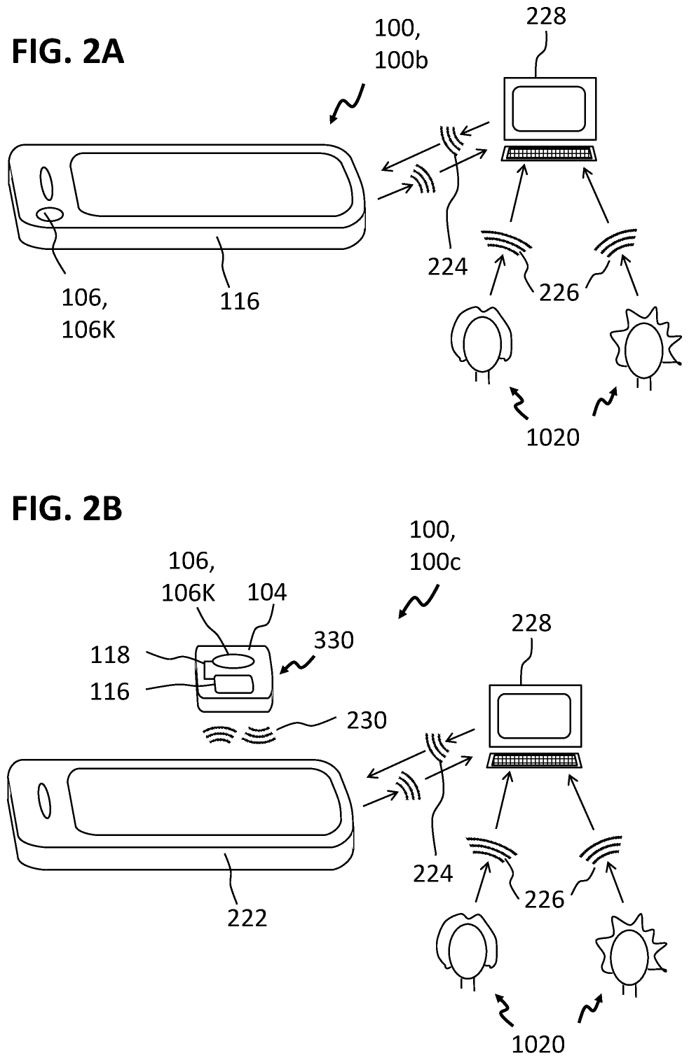

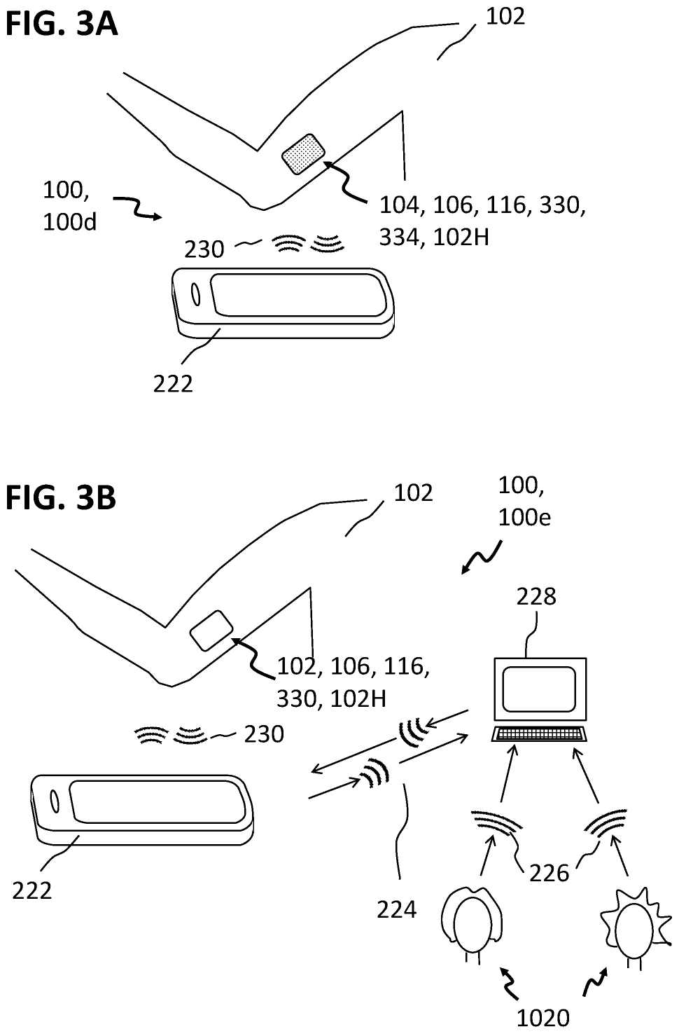

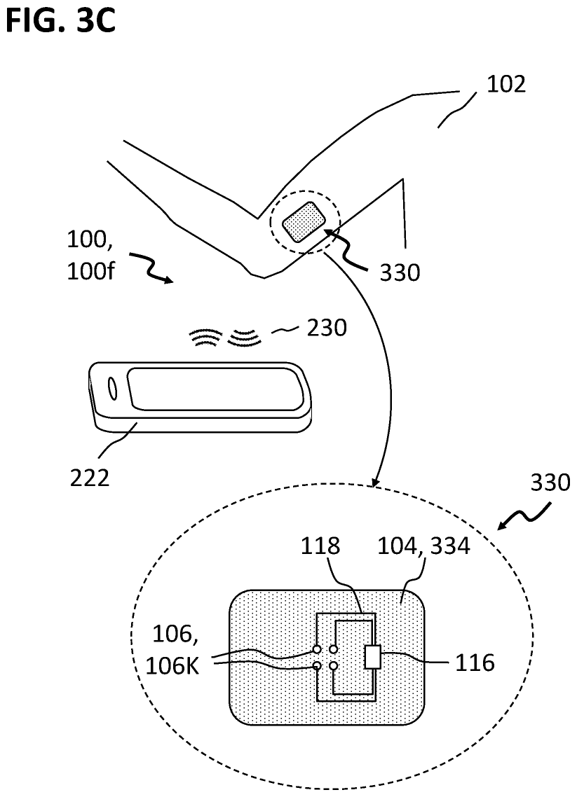

FIG. 1, FIG. 2A, FIG. 2B, FIG. 3A, FIG. 3B and FIG. 3C each show a device 100 (100a, 100b, 100c, 100d, 100e or 1000 for determining a skin moisture content of a user 102 according to various embodiments.

In various embodiments, the device 100 for determining a skin moisture content can have a skin moisture measuring device 106, which can have at least one sensor contact region 106K and can be set up, upon contacting at least one skin region 102H of a user 102, to provide a signal dependent on a skin moisture in the skin region 102H by means of the at least one sensor contact region 102K.

In various embodiments, as described above, the skin moisture measuring device 106 can have at least one of a group of skin moisture measuring devices 106, wherein the group can have a capacitive skin moisture measuring device (also referred to as Corneometer®), an impedance skin moisture measuring device, a skin moisture measuring device utilizing the electrical conductance of the skin and a skin moisture measuring device utilizing transient heat transfer of the skin (also referred to as a TTT meter). Accordingly, in various embodiments, the sensor contact region 102K can have electrodes of a capacitor or applying an AC voltage or probes to introduce heat into the skin and measuring a temperature change. In this case, a basic construction and operation, apart from differences described herein, substantially correspond to that of a corresponding conventional skin moisture measuring device 106.

In various embodiments, the at least one sensor contact region can have a typical area, for example, greater than about 1 mm², for example, in a range from about 1 mm² to about 1 cm² or larger.

In various embodiments, the at least one sensor contact region can have an area of less than about 1 mm². A very small sensor contact region can help to reduce or prevent generation of a weld film between the skin and the sensor contact region of the skin moisture measuring device 106. This can be used in particular when the device for determining the skin moisture 100 is intended to remain for a longer time, as, for example, described elsewhere, for a repeated or continuous measurement of the skin moisture.

In various embodiments, preventing or reducing the accumulation of moisture (perspiration) between the sensor contact region 106K and the skin can enable the moisture content of the skin (for example, the epidermis and the dermis) to be measured and not the moisture on the skin surface, that is, erroneously increased measurements can be avoided.

In various embodiments, by means of a continuous determination of the skin moisture, it is possible to provide checking and follow-up of (development of) the skin moisture, for example, by means of providing the measurement results over time, for example, by means of the software/app, which can display the information on a display, for example, (or can use one of the other options described above, to provide the measurement results to the user 102). Furthermore, in various embodiments, the device 100 can be set up to warn the user of an imminent undersupply of moisture to the skin and to advise on at least one countermeasure, for example, drinking water and/or applying skin care.

In various embodiments, to improve a signal-to-noise ratio, a total sensor contact region (which can be formed as a sum of the sensor contact regions 106K) can be increased by providing a plurality of sensor contact regions 106K spaced apart from each other, as illustrated in FIG. 3C. By way of example, the device 100f has four sensor contact regions 106K, which in a lower, enlarged illustration illustrate a carrier 104 formed as a patch with the sensor contact regions 106 arranged thereon as a top view from below, that is, from a side which faces the skin of the user 102 during a use of the device 100f.

A signal can be provided by means of each of the sensor contact regions 106K, wherein in various embodiments, an average value can be formed from the plurality of signals. In various embodiments, a moisture-absorbing material can be arranged in a spacing region between the sensor contact regions 106K.

In various embodiments, the device 100 can have a carrier 104 on and/or in which the skin moisture measuring device 106 can be arranged, for example, fastened.

In various embodiments, the carrier 104 can be configured to be flexible, for example, as a flexible film, for example, made of a plastic material.

In various embodiments, the carrier 104 may be configured breathable, for example, as illustrated in FIG. 3A and FIG. 3C, can be provided with openings 334 that extend from the skin and/or from above the sensor 106 through the carrier 104 to outside the carrier 104, when the carrier 104 is arranged such that the sensor contact regions 106K are in contact with the skin, wherein the openings can be permeable to steam (for example, water vapor) and/or liquids (for example, water, sweat).

In FIG. 3A to FIG. 3C, the carrier 104 is exemplified as a (flexible) patch which is intended to be fastened to the skin of the user 102 by means of an adhesive, wherein the patches 104 in FIG. 3A and FIG. 3C are formed breathable.

In various embodiments, the device 100 for determining the degree of skin moisture can have an electronic circuit device 116 for detecting the signal provided by the skin moisture measuring device 106 and associating a skin moisture content with the detected signal by means of comparing the detected signal with reference signals for known skin moisture contents. The reference signals (or in general reference data) can be determined in various embodiments, for example, as described above, in laboratory tests and have been updated if necessary by means of additional data of further users 1020, possibly also further data of additional laboratory experiments.

In various embodiments, the database can be stored in an external data processing device 228, for example, a processor cloud architecture 228. The association of the skin moisture content with the detected signal by means of the database stored in the external data processing device 228, for example, the processor cloud architecture 228, can be done directly by the electronic circuit device 116 or indirectly, for example, initiated by the electronic circuit device 116, for example, by the external data processing device 228, for example, the cloud 228.

The electronic circuit device 116 can have a processor and memory in various embodiments. The electronic circuit device 116 can, in various embodiments, be coupled to the skin moisture measuring device 106, for example, such that a detection of a signal provided by the skin moisture measuring device 106 is enabled, for example, by means of a data connection 118. The data connection 118 can have, for example, a data cable, an internal conductive connection and/or a wireless transmission, for example, by means of WLAN, thread, ZigBee or Bluetooth. The electronic circuit device 116 can be, for example, a smartphone, an iPad, a tablet, a laptop, or similar.

In various embodiments, the electronic circuit device 116 can be arranged on and/or in the (for example, breathable) carrier 104.

In various embodiments, the (for example, breathable) carrier 104 can be configured with the skin moisture measuring device 106 and the electronic circuit device 116 as an adhesive patch, as illustrated in FIG. 3A to FIG. 3C. In FIG. 3A and FIG. 3C, the patch can configured breathable by means of the openings 334.

In various embodiments, as shown in FIG. 3B by way of example, the patch can be configured such that moisture-absorbing material is arranged around the at least one sensor contact region 106K (in FIG. 3B covered by the patch), so that the patch can be protected, for example, from penetration of water from the outside, and a generation of liquid film between the sensor contact portion 106K and the skin can still be reduced or prevented. The liquid-absorbing material can also be used in other embodiments, for example, devices 100a, 100b, 100c, 100d, and 100f.

In various embodiments, the device 100 can further have a wireless data interchange device for exchanging data between the electronic circuit device 116 and the processor cloud architecture. This is not illustrated separately in the figures, because it can be formed in various embodiments as part of the electronic circuit device 116. In various embodiments, the wireless data exchange device can alternatively be formed as an independent data exchange device that is connected to the electronic circuit device 116 by means of a data connection.

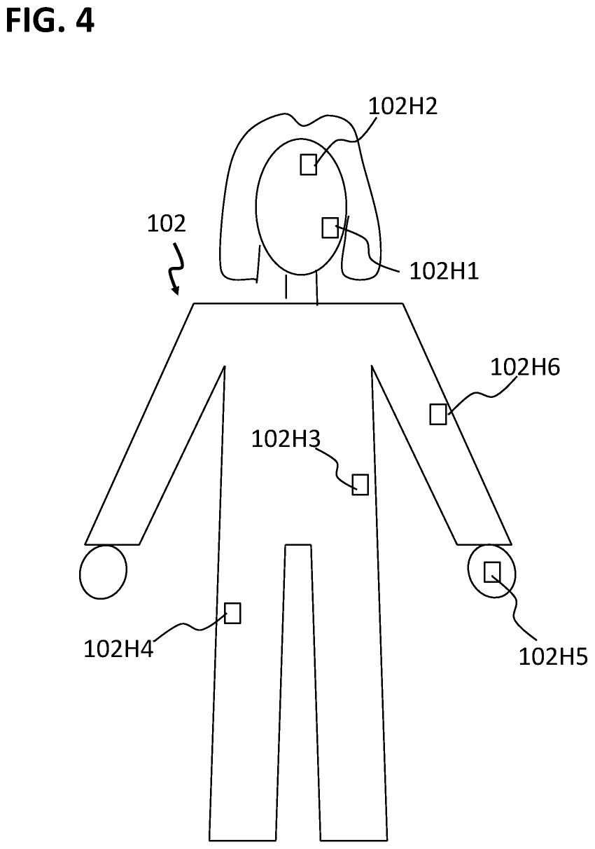

The skin moisture content can be determined in various embodiments for at least one skin region 102H of the user 102. As illustrated in FIG. 1 and as a skin region 102H, in various embodiments, the skin region 102H can be part of a facial skin of the user 102. Alternatively or additionally, as shown in FIG. 4, which shows skin regions 102H for applying method and device for determining a skin moisture content according to various embodiments, another or further skin region 102H, for example, on an arm (skin region 102H6), a for example, (skin region 102H4), a leg (skin region 102H4), a trunk (region 102H4) and/or a hand (region 102H5) of a user 102 can be used to determine a skin moisture content of that skin region.

In various embodiments, the skin moisture measuring device 106 can be part of a wearable device, as illustrated by way of example in FIG. 1, FIG. 2A, FIG. 2B, FIG. 3A, FIG. 3B and FIG. 3C.

In various embodiments, the wearable device can be a smartphone, a tablet, or an iPad, as illustrated in FIG. 2A, or for example, a measuring element 330, as illustrated by way of example in FIG. 2B, FIG. 3A, FIG. 3B and FIG. 3C, wherein a further external data processing device 222 can be part of the device 100, wherein the measuring element 330 can exchange data with the further external data processing device 222, for example, by means of a (for example, wireless) data exchange connection 230. (wherein, alternatively, another form of data transmission can be used, for example, a wired one). In this case, a combination of the integrated unit 330 and the further external data processing device 222 in various embodiments can form the device 100c, 100d, 100e, or 100f for determining the skin moisture content.

In various embodiments, the electronic circuit device 116, which can be part of the measuring element 330, can substantially be provided for detecting the measurement signal provided by the skin moisture measuring device 106 and for providing it to another external data processing device 222, for example, for associating a skin moisture content with the measurement signal. In various embodiments, the wearable device can be attachable to a smartphone, a tablet, or an iPad (not illustrated).

In various embodiments, the detection of the signal and/or the association of the skin moisture content with the detected signal can take place by means of an app.

The electronic circuit device 116 can be set up in various embodiments, as described above, by means of comparing the signal generated by means of the skin moisture measuring device 106, to determine a skin moisture content of the skin of the user 102 (when measuring a plurality of skin regions 102H, the skin moisture content of the respective skin region), for example, a very high, moderately high, normal, moderately low or very low skin moisture content. In various embodiments, the skin moisture content can be otherwise described or quantified, for example, as percent by weight or similarly.

The device 100 for determining skin moisture content according to various embodiments can be a wearable device 100.

In various embodiments (as illustrated in FIG. 2A, FIG. 2B, FIG. 3A, FIG. 3B and FIG. 3C for the devices 100b, 100c, 100d, 100e or 100f respectively), the skin moisture measuring device 106 and the electronic circuit device 116 can form a wearable integrated unit.

In various embodiments, the integrated device 100b, 100c, 100d, 100e, or 100f can have a size that allows them to be accommodated in a hand or trouser pocket. For example, the integrated device can have an area of less than about 36 cm² and a thickness of less than about 2 cm.

The electronic circuit device 116 can be set up in various embodiments to determine a skin moisture content based on the signal measured by the skin moisture measuring device 106. In various embodiments, electronic circuit device 116 can itself be set up, that is, directly, to determine the skin moisture content of the skin.

In various embodiments, for determining the skin moisture content, software, for example, an app, can be utilized, which can be installed, for example, on a wearable data processing device, wherein the wearable data processing device can be, for example, the electronic circuit device 116. For example, in various embodiments, the electronic circuit device 116 of FIG. 1 or of FIG. 2A can be utilized in this way.

In various embodiments, the wearable data processing device can be the further, for example, external, data processing device 222, such as illustrated in FIG. 2B, FIG. 3A, FIG. 3B and FIG. 3C.

In various embodiments, the skin moisture content can be temporally dependent (for example, several times daily, daily, weekly, or with any other temporal dependency) and/or be determined under various environmental conditions (for example, in a high or low humidity environment, cold or heat, etc.).

In various embodiments, calibration measurements (also referred to as reference measurements) that can be performed in a laboratory, for example, and the results of which can be stored in the data processing device, for example, as part of the software/app, can be utilized to associate a signal measurable by means of the skin moisture measuring device, for example, as described above (for example, a voltage, a current or a resistance) with a skin moisture content.

In various embodiments, determining the skin moisture content, for example, as described above, can be carried out directly by means of the wearable electronic circuit device 116, for example, by means of the software/app, which can be installed on the wearable electronic circuit device 116.

In various embodiments, determining the skin moisture content, for example, as described above, can be carried out indirectly by means of the wearable electronic circuit device 116, for example, by causing the electronic circuit device 116 to determine the skin moisture content by means of a further external computing device 222. The electronic circuit device 116, in various embodiments, can have a relatively simple electronic circuit device, which can, for example, be set up to detect substantially only the signal of the skin moisture measuring device 106 (possibly compare it with the reference signals) and to transmit it to the further external data processing device 222. In various embodiments, the electronic circuit device 116 can have a more powerful, versatile circuit device (for example, a smartphone, a tablet, or an iPad) that can not only be set up to detect the signal of the skin moisture measuring device 106 (and possibly compare with the reference signals). and the further external data processing device 222, but can additionally be set up to carry out various further functions, for example, to request and store input from the user 102, to present results, to provide a photograph of the user 102 for displaying the results (for example, by means of a camera) and to process, etc.

In various embodiments, the detected signal and/or the comparison result can be provided by the wearable electronic circuit device 116, as illustrated in FIG. 1 and FIG. 2A, to the external computing device 228, for example, a central computer or processor cloud architecture (also referred to as “cloud” for brevity), for example, be transmitted thereto. The external data processing device 228 can, for example, have a higher computing power and/or a larger storage capacity than the electronic circuit device 116. In addition, the external computing device 228, for example, when configured as a cloud, can enable additional users 1020 to be able to access the external computing device, for example, to update the database. The external data processing device 228 can be set up to determine the skin moisture content from the provided data, for example, the signal or the result of the comparison, for example, by means of a software, for example, an app, for example, as described above by means of comparison with the database. In various embodiments, the electronic circuit device 116 can further be set up to receive and provide the skin moisture content from the further external computing device 222, for example, as described above. In various embodiments, the electronic circuit device 116 can have a device for wireless data transmission device, for example, by means of WLAN, Thread, ZigBee or Bluetooth, for exchanging data with the further external data processing device 222. In various embodiments, the electronic circuit device 116 can be set up to exchange the data with the further external data processing device 222 by means of a cable connection.

In various embodiments, the database can be stored in the wearable electronic circuit device 116, in the external data processing device 228 (for example, the cloud), and/or in the further external data processing device 222.

When determining the skin moisture content, the same skin moisture content can be determined as the skin moisture content of the user for whom the measured value of the skin moisture measuring device 106 shows the smallest deviations from the associated measured value in the database.

In various embodiments, the skin moisture content can be provided by means of the electronic circuit device, for example, by means of the software/app. For example, skin moisture content can be communicated as a value (for example, with arbitrary units), a verbal message, a graphical representation, or similar, for example, displayed, for example, by means of displays, for example, on a screen of the wearable electronic circuit device 116.

In particular, in a case in which the skin moisture content is determined for a plurality of skin regions, providing the skin moisture content to the user can have a graphical representation, for example, a display, for example, by means of a display device (for example, a screen/display). In various embodiments, a representation of a body or a body region (for example, of the face) can be used for this, for example, as a symbolic representation or as a photo of the user 102. The representation can be superimposed on an illustration of the skin moisture content, for example, by means of different symbols/markings/hatching for regions of different skin moisture content.

In various embodiments, the device 100 can further be set up to determine a cosmetic treatment recommendation based on the determined skin moisture content and to provide it to the user 102.

In various embodiments, at least one suitable care product and/or at least one care instruction can be associated with each of the plurality of skin conditions. The association can, for example, be determined experimentally, for example, in laboratory experiments.

In various embodiments, literature data can be the basis of assessing suitability of a care product and/or a care instruction in caring for a skin having a given skin moisture content.

In various embodiments, each of the skin moisture contents may or may not be associated with a quality value. A care product and/or a care instruction can be rated as suitable for a skin moisture content when it is to be expected, for example, on the basis of literature data, test results or empirical values, that with a (for example, regular) application of the care product and/or the care instruction, the skin moisture content of the user 102 is maintained or changed to a skin moisture content having a higher quality value (for example, toward a normal skin moisture content).

In various embodiments, an assessment of a care product's suitability to improve a skin moisture content can be confirmed or altered by means of receiving empirical values of further users 1020 having the same or similar skin moisture content, for example, empirical values with respect to a treatment success similar to what is illustrated in FIG. 1, FIG. 2A, FIG. 2B and FIG. 3B for updating the database stored in the processor cloud architecture 228 to determine the skin moisture content by the further users 1020. The empirical values can be provided by the further users 1020, for example, to the external data processing device 228, for example, by means of a wireless data transmission 226. Alternatively, a transmission of the data by means of cable can be used. The database in the further external computing device 222 and/or in the wearable electronic circuit device 116 can be updated on the basis of empirical values. It is thus possible for the user 102 to always receive an optimal recommendation.

In various embodiments, when determining the product and/or care recommendations, further information regarding a general health condition, a skin condition, dietary habits, and further user behaviors (for example, daily exposure to sun/water, smoking habits, sports habits, etc.) can be used, for example, by means of the software/app installed on the wearable electronic circuit device 116 and/or on the further external data processing device 222. The information can, in various embodiments, be queried by the user 102 by means of the wearable electronic circuit device 116, which can enter it into the electronic circuit device 116, for example, by means of a keyboard, as a voice message, as a selection from a menu shown by the wearable electronic circuit device 116, or similar.

For example, when the user 102 provides as additional information that he spends much time in the water or outdoors, the user 102 receives recommendations which are not only suitable for his skin moisture content, but also, for example, waterproof and/or provided with a UV filter for the product and/or care instruction recommendation based on the skin moisture content of the user 102, for example, those associated care products.

In various embodiments, the product recommendation and/or the care instruction can be provided to the user 102, for example, by means of the electronic circuit device 116, for example, by means of the screen of the electronic circuit device 116. For example, a graphical representation can be supplemented by (for example, text) messages, for example, the determined product recommendations and/or care instructions.

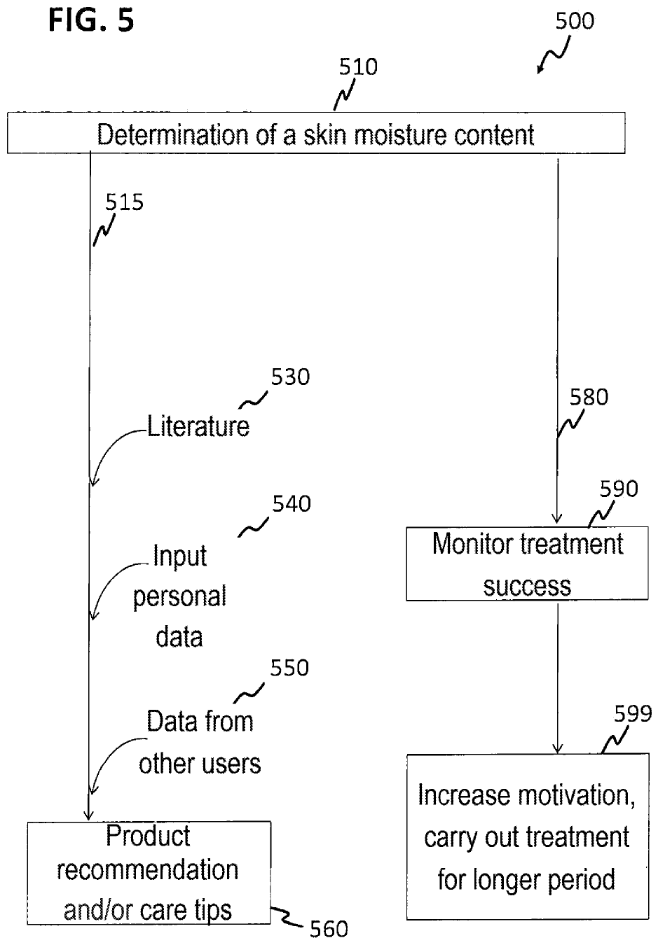

FIG. 5 shows a diagram 500 illustrating a method for determining a cosmetic product for skin treatment according to various embodiments. FIG. 5 can substantially illustrate those processes described elsewhere in the context of the method for determining a cosmetic product for skin treatment according to various embodiments.

The method can have, in various embodiments, a determination of a skin moisture content (at 510), for example, as described above.

A product or care recommendation can be determined (at 560) based on the calculated skin moisture content. In various embodiments (labeled as path 515), in addition to the skin moisture content, literature data (labeled 530), personal data (for example, as described above with 540), and/or data of further users, for example, their empirical values (labeled 550) can be called upon.

In various embodiments, furthermore, as illustrated at path 580, a treatment success can be monitored (at 590) by means of determining the skin moisture content (at 510). This can be useful, for example, especially during and after a cosmetic treatment based on the product recommendation (at 560). A treatment success documented by means of the method for determining the skin moisture content on the basis of objective values can increase a motivation for the user (at 599).

In various embodiments, as described above, product recommendations for cosmetic products that are individually adapted to the user 102 and/or individual care instructions can be derived on the basis of the determined skin moisture content. The product recommendations and/or care instructions can be provided, for example, by means of software, for example, an app.

The user 102 can be offered a recommended cosmetic product directly for purchase and the user 102 can initiate the purchase via an entry. In addition to the purchase of recommended cosmetic products, the user can also be offered further information on the purchase. This more detailed information can refer to more detailed care instructions. For example, a software/app receives the request that the user 102 would like to purchase the cosmetic product, stores the request, and/or submits the request to a retailer that distributes the cosmetic product. The user 102 can be prompted by the software/app to enter his personal data (address, bank information, delivery preference, etc.) via the input unit. Alternatively, the user received information on where (for example, drugstore, pharmacy, etc.) he can purchase the oral hygiene product.

In various embodiments, the user 102 can be encouraged to use cosmetic products that are manufactured individually for the user and can initiate an ordering process, preferably by accessing a manufacturer's website of individual cosmetic products.

More and more users are looking for a product that is individually tailored to their needs. This can be a product manufactured especially for the user or a so-called “mass customized” product. In the case of a “mass customized” product, an individualization can be achieved by varying a few characteristics of a product that are decisive from the user's point of view. Preferably, these “mass customized” products are based on the concept of modularization, that is, the product can be assembled individually from various modules/building blocks.

Often, there are many dependencies between the many different features/ingredients of a product, which can be expressed as “commandments” or “prohibitions.” In order to obtain a clear product definition, it can be advantageous for the ordering process to proceed with the aid of a product configurator. This configurator helps the user to select the characteristics/ingredients and draws attention to the permitted/inadmissible combinations of features, wherein the latter then can not be selected.

With products for the skin, the relevant product characteristics comprise, in particular, the chemical ingredients of the agent, the physical properties of the agent and the manner of preparation of the agent. With the help of a product configurator, for example, the selection of chemically and/or physically incompatible ingredients or the selection of the ingredients unsuitable for the determined skin moisture content can be avoided. Conversely, the selection of suitable ingredients for the determined skin moisture content can be predetermined or suggested by the product configurator.

In various embodiments, the software/app that determines the skin moisture content of the skin can be the same as that which determines the product recommendation and/or the care instruction and/or initiates the ordering process. In various embodiments, different software programs/apps can be used for a part of the various operations or all of the various operations (determining the skin moisture content, determining a product recommendation, determining a care instruction, initiating a ordering process).

In various embodiments, visiting a dermatologist or a cosmetician can be recommended. In various embodiments, a booking process can be initiated directly via the software/app, which determines the skin moisture content and/or the product recommendation and/or the care instruction and/or initiates an ordering process. For this purpose, for example, the contact data of dermatologists and/or cosmeticians can be stored in the software/app and these can be displayed to the user. In addition, the selection can be restricted via filters, such as the postal code. In various embodiments, an appointment booking can be made directly via the software/app. Alternatively, the booking of a dermatologist appointment and/or a cosmetic appointment can be made via a separate software/app, such as Treatwell.

In various embodiments, a treatment success in a cosmetic treatment, which can have as a goal a positive effect on the determined skin moisture content, can be monitored. In various embodiments, the software/app can enable control and/or tracking of the results by means of a representation (for example, a graphical representation) of the measurement results over time.

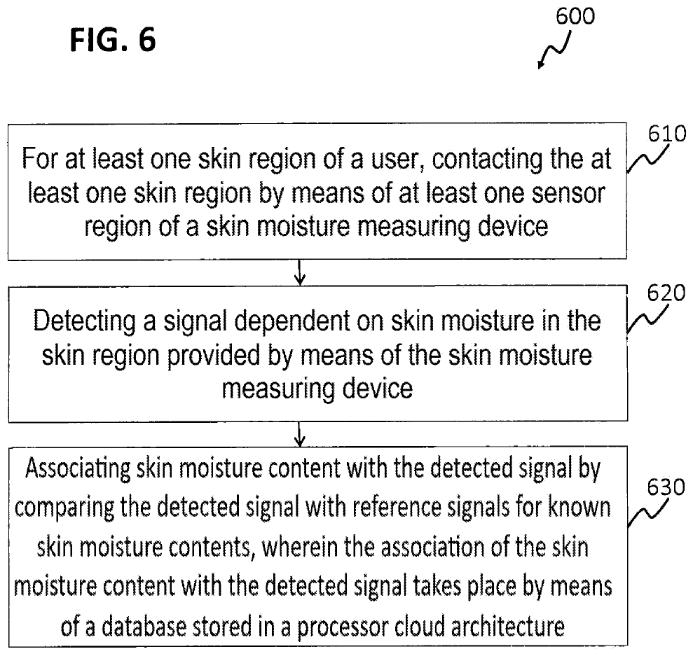

FIG. 6 shows a flowchart 600 of a method for determining a skin moisture content according to various embodiments.

In various embodiments, a method for determining a skin moisture content is provided. The method can have a contacting of the at least one skin region by means of at least one sensor region of a skin moisture measuring device for at least one skin region of a user (at 610), detecting a signal (at 620) dependent on a skin moisture in the skin region and provided by means of the skin moisture measuring device, and associating a skin moisture content with the detected signal by means of comparing the detected signal with reference signals for known skin moisture contents, wherein the association of the skin moisture content with the detected signal takes place by means of a database stored in a processor cloud architecture (at 630).

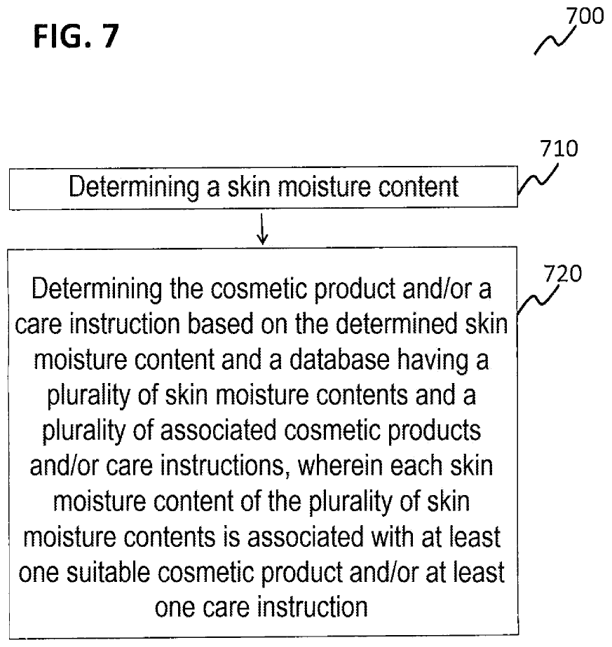

FIG. 7 shows a flowchart 700 of a method for determining a cosmetic treatment recommendation according to various embodiments.

In various embodiments, a method for determining a cosmetic treatment recommendation is provided. The method can have: a determining of a skin moisture content according to various embodiments (at 710) and a determining of the cosmetic product and/or the care instruction based on the determined skin moisture content and a database having a plurality of skin moisture contents or a plurality of associated cosmetic products/care instructions, wherein each skin moisture content of the plurality of skin moisture contents can be associated with at least one suitable cosmetic product and/or at least one suitable care instruction (at 720).

Some of the embodiments are described in the context of devices, and some of the embodiments are described in the context of methods. Further advantageous embodiments of the method are apparent from the description of the device and vice versa.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for determining a skin moisture content, the method comprising:
contacting at least one skin region with at least one sensor contact region of a skin moisture measuring device;
detecting a signal dependent on skin moisture in the skin region with the skin moisture measuring device; and
associating a skin moisture content with the detected signal by comparing the detected signal with reference signals for known skin moisture contents, wherein the reference signals are located in a database stored in a processor cloud architecture.

2. The method of claim 1, further comprising:
updating the database based on new associations of the skin moisture content with the detected signal from a plurality of further users.

3. The method according to claim 1,
wherein the skin moisture measuring device has at least one of a group of skin moisture measuring devices, wherein the group includes:
a capacitive skin moisture measuring device;
an impedance skin moisture measuring device;
a skin moisture measuring device utilizing the electrical conductance of the skin, and a skin moisture measuring device utilizing a transient heat transfer of the skin.

4. The method according to claim 1,
wherein after contacting the at least one skin region with the at least one sensor contact region of the skin moisture measuring device, detecting the signal dependent on skin moisture in the skin region with the skin moisture measuring device includes detecting the signal dependent on skin moisture in the skin region with the skin moisture measuring device several times at a time interval or continuously.

5. The method according to claim 1,
wherein the at least one sensor contact region has a plurality of sensor contact regions spaced apart from each other, and wherein the method further includes:
forming a signal average value from the plurality of signals of the plurality of sensor regions.

6. The method according to claim 1,
wherein the at least one skin region has a plurality of skin regions of the user.

7. The method according to claim 1,
wherein the skin moisture measuring device is part of a wearable device.

8. A method for determining a cosmetic skin treatment recommendation, the method comprising:
determining a skin moisture content according to claim 1; and
determining the cosmetic product and/or a care instruction based on the determined skin moisture content and a further database stored in the processor cloud architecture having a plurality of skin moisture contents and a plurality of associated cosmetic products and/or care instructions, wherein each skin moisture content of the plurality of skin moisture contents is associated with at least one suitable cosmetic product and/or at least one care instruction.

9. The method according to claim 8,
wherein the cosmetic product and/or the care instruction is suitable for the skin moisture content, when a normalization of the skin moisture content is to be expected based on empirical values stored in the further database.

10. The method according to claim 8,
wherein determining the skin moisture content and/or determining the cosmetic skin treatment recommendation has a transmitting of the signal and/or the skin moisture content to an external data processing device and a receiving of the skin moisture content and/or the cosmetic skin treatment recommendation.

11. A device for determining a skin moisture content, the device comprising:
a skin moisture measuring device having at least one sensor contact region and configured to provide a signal dependent on a skin moisture in the skin region by contacting eat least one skin region of a user with the at least one sensor contact region;
an electronic circuit device configured for detecting the signal provided by the skin moisture measuring device and associating a skin moisture content with the detected signal by comparing the detected signal with reference signals for known skin moisture contents, wherein association of the skin moisture content with the detected signal is performed with a database stored in a processor cloud architecture.

12. The device according to claim 11,
wherein the skin moisture measuring device has at least one of a group of skin moisture measuring devices, wherein the group includes:
a capacitive skin moisture measuring device;
an impedance skin moisture measuring device; a skin moisture measuring device utilizing the electrical conductance of the skin; and
a skin moisture measuring device utilizing a transient heat transfer of the skin.

13. The device according to claim 11, further comprising:
a breathable carrier, wherein the skin moisture measuring device and the electronic circuit device are arranged on the breathable carrier.

14. The device of claim 11, further comprising:
a wireless data interchange device arranged on the breathable carrier for exchanging data between the electronic circuit device and the processor cloud architecture.

15. A device for determining a skin moisture content, the device comprising:
a breathable carrier;
a skin moisture measuring device arranged on the breathable carrier, wherein the skin moisture measuring device includes at least one sensor contact region configured to provide a signal dependent on a skin moisture in the skin region by contacting of at least one skin region of a user with the at least one sensor contact region; and
an electronic circuit device arranged on the breathable carrier for detecting the signal provided by the skin moisture measuring device and for associating a skin moisture content with the detected signal by comparing the detected signal with reference signals for known skin moisture contents.
